(19) United States
(12) Patent Application Publication
(10) Pub. No.: US 2004/0137606 A1
(43) Pub. Date: Jul. 15, 2004

(54) Programmable mask for forming biomolecule or polymer array and fabrication method of biomolecule or polymer array using the same

(76) Inventors: Moon Youn Jung, Daejon-Shi (KR); Dong Ho Shin, Daejon-Shi (KR); Hae Sik Yang, Daejon-Shi (KR); Yong Shin Kim, Daejon-Shi (KR); Tae Hwan Yoon, Kyeonggi-Do (KR); Chi Hoon Jun, Daejon-Shi (KR); Yun Tae Kim, Daejon-Shi (KR); Seung Youl Kang, Daejon-Shi (KR); Yong Eui Lee, Seoul (KR); Seong Deok Ahn, Daejon-Shi (KR); Kyung Soo Suh, Daejon-Shi (KR)

Correspondence Address: Jacobson Holman, 400 Seventh Street, N.W., Washington, DC, 20004-2218, US

(21) Appl. No.: 10/703,553

(22) Filed: Nov. 10, 2003

(30) Foreign Application Priority Data
Dec. 11, 2002 (KR) 2002-69882

(51) Int. Cl. C12M 1/34; B05D 3/00

(52) U.S. Cl. 435/287.2; 427/2.11

(57) ABSTRACT

The present invention relates to the transmissive programmable mask for forming biomolecules such as DNA or polymer array by irradiating specific cells with incident lights, and to the method for forming the biomolecules or polymer array using the same.

Each unit pixel of the programmable mask comprises, a solution which includes charged particles which are moved by electrophoresis and interrupt the progress of the incident light, and electrodes for applying voltages to the particles in order to adjust the transmissivity of the incident light by changing the arrangement of the particles.

### BACKGROUND OF THE INVENTION

1. Field of the Invention

The present invention relates to a programmable mask for forming biomolecule or polymer array, in particular, to a transmissive programmable mask for forming biomolecule or polymer array by irradiating a certain cell with UV lights and method for fabricating biomolecule or polymer array using the same mask.

2. Description of the Prior Art

First, an area consisted of one kind of biomolecules or polymer within a biomolecule array formed on a substrate is referred to as a cell.

Researches have been conducted over tasks that perform several kinds of experiments at one time using an array f biomolecules or polymers Examples of the array of biomolecules or polymers can include an array of polypeptide, carbohydrate, nucleic acid (DNA, RNA), etc. In order to conduct such researches, it is most important to effectively form an array with high purity on a substrate at a low cost.

To date, there have been several methods for forming biomolecule or polymer array, for example a spotting method that a micro-robot selectively drops biochemical material on a required position while it moves in three dimensional directions, an electronic addressing method that biomolecule are fixed to a specific electrode by adjusting an electrode voltage of a microelectrode array, and a photolithography method that bonding reaction of cell surface and biomolecule is occurred in specific position by selectively irradiating a required position of the cell with light thereby deforming the surface of the required position. The spotting method is classified into a contact printing method that spots the solution like stamping on a paper and a non-contact printing method that drops the solution. The contact printing method performs loading, printing and washing in this order by means of a XYZ robot. As the robot uses the pin having a groove at the end thereof like a tip of a fountain pen, this method can reproductively adjust sample volume and perform printing several times once it has loaded the sample. However, it cannot greatly increase the number of array per unit area.

The non-contacting method is classified into a dispensing one and an ink-jet printing one. The dispensing method drops a solution, which is similar to the use of a micropipette, and the ink-jet printing method has the solution to be spouted by applying pressure to an ink reservoir. If the ink-jet printing method is used, the sample solution can be micro adjusted up to a nano liter level thereby the number of an array per unit can be increased. An ink reservoir is needed per sample solution, and however, the number of ink reservoir that can be mounted to a robot is limited, so that this method for forming an array can be used only when a few sample solutions are used.

The electronic addressing method fixes the biomolecule by using the function of voltage adjustment of a microelectrode array, and is classified into a method that makes the charged biomolecule move to an electrode surface to being physichemically bonded, and a method that fixes the biomolecule within a thin film formed by performing electrochemical deposition. (See Cosnier, Serge, “Biomolecule immobilization on electrode surfaces by entrapment or attachment to electrochemically polymerized films. A review” Biosensors & Bioelectronics 14: pp. 443-456, 1999). For example, As DNA is strongly negative charged, it moves toward an electrode when the electrode is positively charged. The DNA is fixed to the electrode when a physiochemical bond is occurred between the DNA and the electrode. (See the following publication that is incorporated herein by reference: U.S. Pat. No. 5,605,662) Such electronic addressing method cannot be applied to a large number of arrays, and has a disadvantage that it basically needs a microelectrode. And, the method has also been developed which selectively fixes the biomolecule to a required position by electrochemically changing pH around an electrode, and Combimatrix Co. has disclosed the method using the concept that fixes an oligonucleotide to a required position of a microelectrode. (See the following publication that is incorporated herein by reference: U.S. Pat. No. 6,090,302) This method has a disadvantage that the purity of each cell is greatly low due to a low yield of each reaction.

Meanwhile, Affymetrix Co. used the photolithography method for the first time that has been used in a semiconductor process. (See the following publication that is incorporated herein by reference: U.S. Pat. No. 5,959,098) This method has an advantage that it can make an array with high density and perform parallel synthesis. However, it requires many costs and time, as many photomasks are needed.

The method has also been developed which uses the programmable mask that can adjust the path of light per each pixel without any photomasks. (See the following publication that is incorporated herein by reference: U.S. Pat. No. 6,271,957) The programmable mask uses the method for adjusting the reflection of light, or the method for adjusting the transmission of light, and one example for the reflection includes the method using a micromirror array, and one example for the transmission includes the method using a LCD (liquid crystal display). The method for forming an array by means of adjustment of the micromirror array requires a very complicated optical system, and has a disadvantage that it can only obtain mosaic patterns. (See the following publications that are incorporated herein by references: U.S. Pat. Nos. 6,271,957 and 6,375,903)

The transmissive programmable mask relatively needs a simpler optical system than that of a reflective programmable mask, however, cannot greatly increase the contrast ratio that corresponds the transmission rate of optical transmission to optical non-transmission. When the contrast ratio is low, since UV light will transmit even through the unrequired pixels to cause a photoreaction occurs at a cell, biomolecules or polymer array having high purity cannot be obtained. In the LCD type programmable mask, optical transmission of each pixel is adjusted thereby photoreaction occurs to the corresponding cell on a substrate and this process is repeated, so that the biomolecule or polymer array is formed. (See the following publications that are incorporated herein by references: Korea Patent Application No. 2001-0002915 and U.S. Pat. No. 6,271,957) However, the LCD type programmable mask has a disadvantage that it has a low UV transmissivity and is deformed by the UV. In a general LCD, the LCD type programmable mask acts to transmit the visible light, however, needs the light having 400-330 nm in wavelength in order to detach protective group. Liquid crystal and orientation film that are used for the general LCD tend to be deformed by the UV light.

Meanwhile, Researches over adjusting the optical transmission have been conducted in a display field. In particular, the electronic ink technique has been developed for moving charged particles by means of electrophoresis thereby changing light color. (See the following publication that is incorporated herein by reference: U.S. Pat. No. 6,120,588) This technique changes light color that is reflected, so that it can not be used for the transmissive purpose. Since the technique also has a low contrast ratio, it is not suitable for forming the biomolecule or polymer array.

Therefore, in order to form biomolecule or polymer array having high density, a programmable mask having high density is needed and can be obtained by controlling the transmissivity of each pixel by means of transistors, as it is performed in a general display field.

### SUMMARY OF THE INVENTION

Therefore, the object of the present invention is to provide a novel programmable mask capable of forming biomolecules or polymer array having high density in easy and low cost way.

To achieve the above object, according to one aspect of the present invention, A programmable mask is provided for forming biomolecules or polymer array, which includes an array of unit pixels and a driver circuit for selectively applying a voltage to each of said unit pixels to adjust the transmissivity of an incident light for each unit pixel, each of which comprises: a solution which includes charged particles which are moved by electrophoresis and intercept a progress of the incident light; and electrodes for applying an voltage to the particles in order to adjust the transmissivity of the incident light by changing the arrangement of the particles.

“The programmable mask” means all devices capable of controlling transparency/opacity of an incident light per unit pixel. And if needed, the mask can be configured for the gray level as well as the transparency and opacity level. Meanwhile, switching devices for selectively applying a voltage to unit pixel are not limited to a thin film transistor, MIN element, etc and can be any devices that can perform switching. A driver circuit can be implemented on a substrate, together with the pixels, or can be separately implemented on a printed circuit board.

In addition, the solution can be a suspending fluid consisting of a fluid and charged particles, and the fluid comprises a fluorocarbon, chlorocarbon, fluorochlorocarbon, or a trichlorofluoroethylene, and the charged particles are TiO₂ having 500˜3000 Å in size. Meanwhile, the charged particles can intercept the transmission of UV light by absorbing or reflecting the UV light.

Preferably, the charged particles are collected on selected electrodes by the voltage difference of said electrodes, and the incident light is intercepted when the charged particles are collected on the electrodes vertical to the incident direction of the incident light, and the incident light is transmitted when the charged particles are collected on the electrodes parallel to the transmissive direction of the incident light, and when the difference voltage of the electrodes does not exists, the charged particles are distributed at random, thereby the incident light is intercepted.

The another aspect of the present invention comprises a method for forming biomolecules or polymer array using the programmable mask as explained above, and the method comprises steps of (a) irradiating selected area of the molecules which are fixed on the surface of the mask and have protective group with UV lights using the programmable mask; and (b) having a solution containing the biomolecules or polymer monomer which needs to be fixed flow into the selected area of the molecules.

### BRIEF DESCRIPTION OF THE ATTACHED DRAWINGS

FIG. 1 is a concept view for explaining photoreaction using the transmissive programmable mask used in the present invention.

FIG. 2 is a plane view of the transmissive programmable mask used in to the present invention.

FIG. 3 is a concept view of transistors for adjusting transmissivity of pixels configuring the transmissive programmable mask.

FIG. 4 is a concept view for optical transmission and non-transmission using particles of the present invention.

FIG. 5 is a concept view for intercepting UV transmission of each pixel forming the transmissive programmable mask.

FIG. 6 is a concept view of method for obtaining UV transmission of each pixel forming the transmissive programmable mask.

FIG. 7 is a concept view for an array of the pixels forming the transmissive programmable mask.

FIG. 8 is a spectrum of UV transmissivity when a suspension fluid containing TiO₂ with charged particles exists between quartz substrates and when the suspension fluid doest not exist.

### DESCRIPTION OF THE PREFERRED EMBODIMENT

The purpose and several advantages of the present invention will be made more apparent from the preferred embodiment with reference to the accompanying drawings by those who are skilled in the art.

Hereinafter, the preferred embodiment of the present invention will be explained with reference to the accompanying figures.

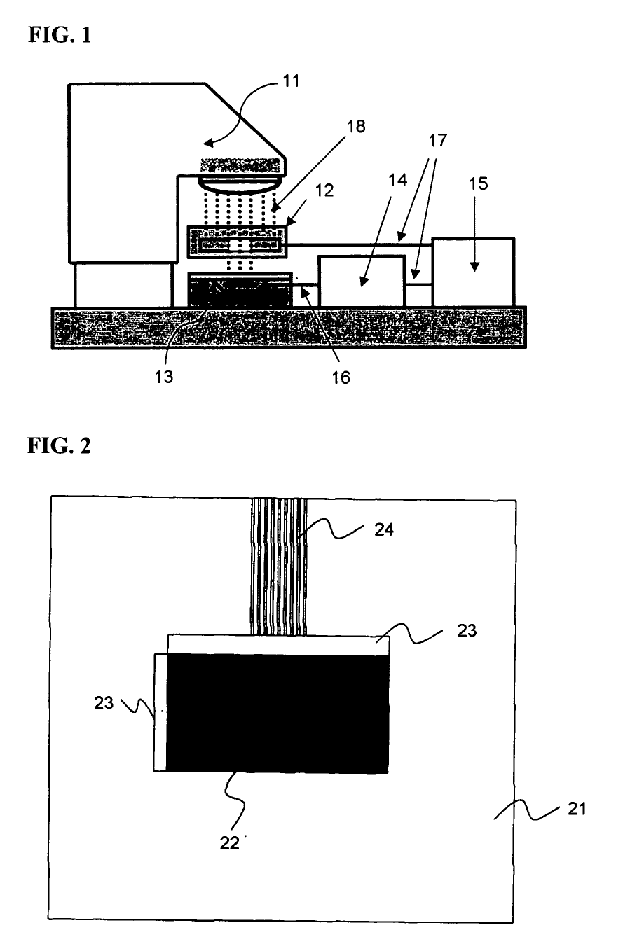

FIG. 1 is a concept view for explaining photoreaction using the transmissive programmable mask used in the present invention. The UV light 18 emitted from the UV light source 11 passes through the transmissive programmable mask 12, and a light transmission area of the mask is determined by the signals which are programmed in the computer 15 and transferred through the cable 17. At the microreactor 13 which has a fluid delivery device (not shown) connected to the substrate in which DNA array or polymer array is formed, the UV light 18 which has passed through the programmable mask is irradiated on the array substrate, thereby photoreaction occurs. The microreactor 13 is connected to the DNA synthesizer 14 controlled by the computer 15, and is supplied with samples needed for forming the array and supplied with a washing solution needed for washing the substrate, through the tube 16.

FIG. 2 is a plane view for transmissive programmable mask used in the present invention. An irradiating area 22 of the UV light is formed in a substrate 21 like a quartz which has a high transmissivity of the UV light, and a driving IC area which applies an electrical signal and selects the pixel which the light will pass through by applying an electrical signal is placed around the area 22, and an electrode pad 24 is placed to connect the driving IC to an external IC. Transmissivity of pixels is determined by the array data from the computer, and this procedure is repeated to obtain the DNA or polymer array having required arrangement.

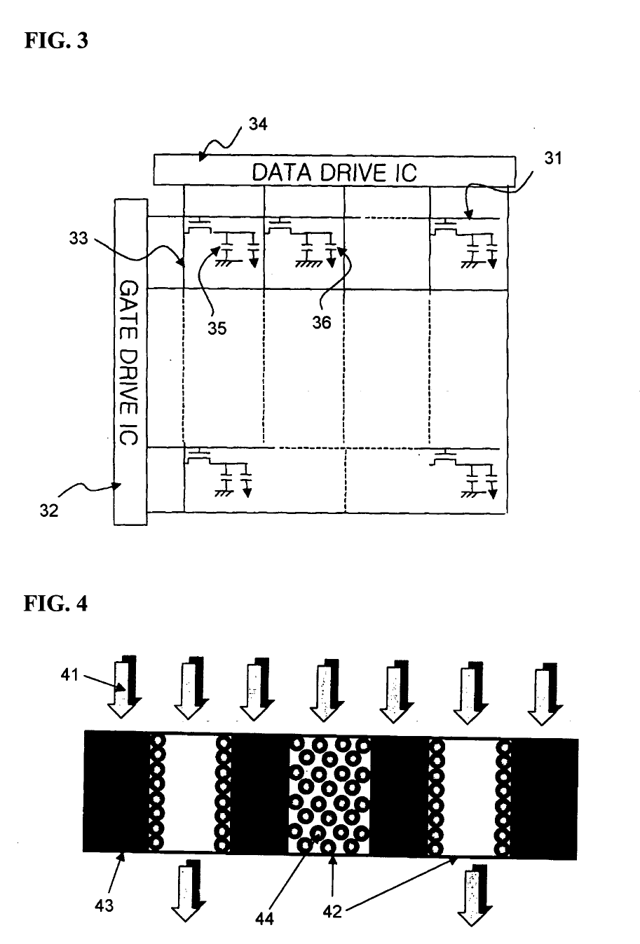

FIG. 3 is a concept view for transistors adjusting transmissivity of pixels forming the transmissive programmable mask. Silicon thin film transistors are commonly used for the transistors, each thin film transistor is connected to the gate wiring 31, data wiring 33, and the storage electrode 36 which is parallelly connected to the capacitor 35, and supports the capacity of the capacitor 35 while each pixel is switched on. The gate wiring 31 and the data wiring 33 are electrically connected to a driving IC. Each gate wiring 31 is sequentially selected, and whenever the each gate wiring 31 is selected, a switching signal is input to the data wiring 33, to perform ON/OFF switching is performed for transmissivity of each pixel. When switching operation for all pixels are completed, a required transmission pattern for a total array can be obtained.

FIG. 4 is a concept view for optical transmissivity and non-transmissivity using particles in accordance with the preferred embodiment of the present invention. In the programmable mask consisting of three pixels in FIG. 4, each pixel consists of an area 42 in which transmission of the UV light 41 is adjusted and an area 43 in which the UV light 41 is intercepted. Devices like transistors needed to control the light are placed in the area 43. The UV light is passed through when the particles 44 intercepting the progress of the light are placed toward side walls, and is intercepted when the particles 44 are dispersed. To facilitate the control of the particles 44, the particles 44 are charged and placed within a solution. The charged particles can be placed near electrodes formed on the side walls by an electrophoresis, or can be dispersed within the solution by applying no voltage to the electrodes. To facilitate the easier control of the particles 44, it is preferable that the particles have a plurality of charges and be placed within a suspending fluid. In addition, in order to effectively intercept the UV light, the particles 44 should absorb or reflect the UV light. These particles 44 may not be easily melt in a solution, or may be consisted of small ones of a molecule level.

In the suspending fluid consisting of a fluid and a plurality of charges, the fluid has to easily allow the light to transmit, so that the solution that easily absorbs the UV light is not suitable for the fluid. Therefore, it is preferable to use the fluid that can easily transmit the UV light and has a good stability for the UV light, and a fluorocarbon, chlorocarbon, fluorochlorocarbon, trichlorofluoroethylene, etc can be used for the fluid.

TiO₂, barium sulfate, kaolin, zink oxide, etc can be used for the charged particles having 500-3000 Å in size.

In addition, dispersing agents can be added to the solution, and the examples of the dispersing agents may comprise ethylene glycols as charge adjuvents, a polyhydroxy compound capable of containing polypylene glycol, 3-amino-1 propanol, an amino alcohol compound capable of containing triethanolamine. Furthermore, surface modifiers or charge control agents can be added to the solution.

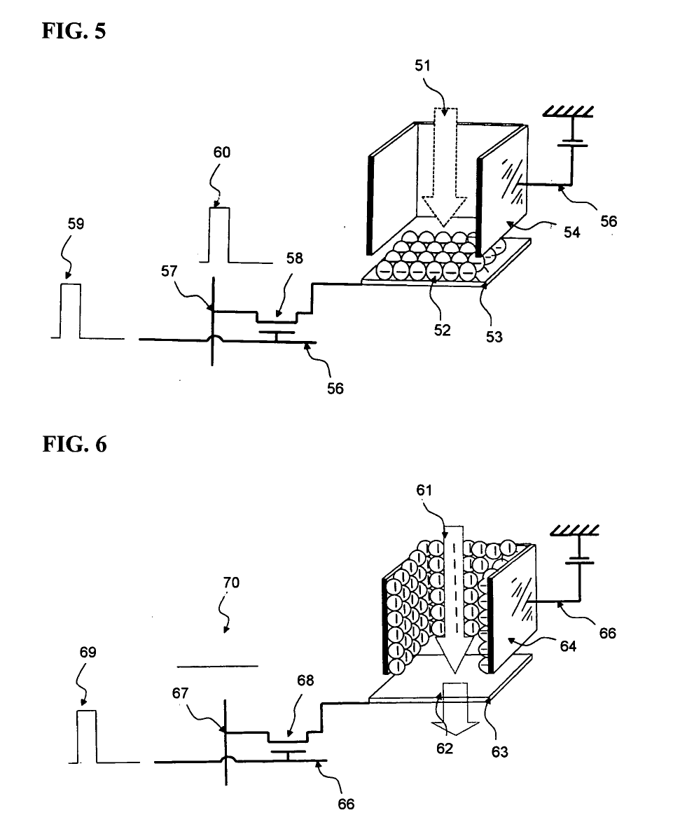

Meanwhile, in the case that charged particles are not easily melt to hydrophobic solvent, the UV light can be intercepted by aligning the charged particles capable of intercepting the UV light being vertical to the progress direction of the light while using different electrodes from those of FIG. 5. In this case, the charged particles can be intercepted by applying a voltage to the transparent electrode, such as ITO (Indium tin oxide), which has been formed in the transparent direction of the UV light. Referring to FIGS. 5 and 6, the UV light is intercepted when the particles 52 are collected on the transparent electrode 53 vertical to the progress direction of the UV light 51 by electrophoresis as shown in FIG. 5, and is transmitted when the particles 62 are collected on the transparent electrode 63 parallel to the progress direction of the UV light 61 by electrophoresis as shown in FIG. 6. The voltage of the electrode 54 vertical to the electrode 53 can be adjusted by a transistor.

In order to determine transmission or non transmission of one pixel by one transistor, the voltage of the vertical electrode 54 is fixed while the voltage of the parallel electrode 53 is adjusted by the transistor. Half of the maximum voltage is applied to the vertical electrode 54, and a ground or a maximum voltage is applied to the parallel electrode 53, so that the particles charged by the voltage difference between the vertical electrode and the parallel electrode are moved by electrophoresis. Although the parallel electrode 53 is placed below the vertical electrode 54 in the embodiment of the present invention, the parallel electrode can be placed above or at both sides of the vertical electrode 54 in accordance with the kinds of the parallel electrode 53, vertical electrode 54, charged types, and voltages applied to the electrodes.

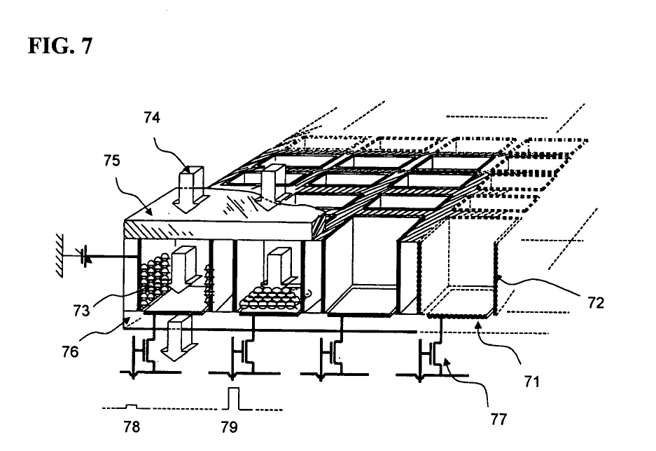

FIG. 7 is a concept view for an array of the pixels forming the transmissive programmable mask. Each transistor is connected to each pixel, and two electrodes are placed in a space containing a solution through which the UV light 74 transmits, and charged particles 73 are collected on the electrodes 71 vertical to the progress direction of the UV light or on the electrodes 72 parallel to the progress direction of the UV light. Upper substrate 75 and lower substrate 76 are cohered to prevent the solution from being leaked. Meanwhile, FIG. 7 shows a gate voltage 79 that switches on the transistor of an unit pixel and of a gate voltage 78 that switches off the transistor.

Meanwhile, as described above, in order to confirm the interception of the UV light, one UV spectrum was obtained when the suspending fluid was existed and another when the suspending fluid was not. FIG. 8 shows the result of the transmissivity measured by changing the wavelength of the UV light in a UV wavelength range, while the suspending fluid is injected between quartz substrates with 0.6 mm in thickness in which 1% of the TiO₂ having 500-3000 Å in size is contained to a trichlorofluoroethylene solution with dispersing agents, compared to the transmissivity of the only quartz substrate. In the 340-370 nm range known for the most suitable UV wavelength range for the DNA array fabrication, the transmissivity of the sample with the suspending fluid injected is about 0.5%, and the transmissivity of two quartz substrates is about 98˜99%, so that the difference of the transmissivities (i.e. contrast ratio) is about 150:1. The more the amount of the suspending fluid increases, the greater the contrast ratio increases.

Hereinafter, the method for forming biomolecule or polymer array using the programmable mask described above.

First, the UV light is irradiated on a selected area of the molecule having a protecting group and being fixed on the surface of the mask by using the programmable mask, so that the protecting group is felt apart from the molecule and OH basic is exposed. And the biomolecules or polymer monomer is fixed on the only exposed OH basic portion if a solution containing the biomolecules or polymer monomer that needs to be fixed is flowed to the exposed portion. As the polymer monomer fixed from the above process has another protecting group, another monomer can be fixed if a selected area of the molecule is irradiated, and if this procedure is repeated, then biomolecules or polymer array having required arrangement can be obtained.

### EFFECT OF THE INVENTION

According to the present invention as described above, biomolecule such as DNA or polymer array can be fabricated by adjusting the transmission of UV light.

The present invention has an advantage that can form a high density array in a much easier and cheaper way than the method using a conventional optical mask, micromirror array, and LCD. As high contrast ratio can be obtained, high purity biomolecule or polymer array can be fabricated. And, when stepping function exists or several patterns exist for one programmable mask, mass production of biomolecule or polymer array can be easily obtained.

In addition, the programmable mask according to the present invention can be manufactured in small size, so that it can be applied for general purpose DNA chip manufacturing device which can be used at a hospital and a laboratory. Therefore, high density DNA chips can be manufactured at a low cost.

What is claimed is:

1. A programmable mask for forming biomolecules or polymer array, the mask including an array of unit pixels and a driver circuit for selectively applying a voltage to each of said unit pixels to adjust the transmissivity of an incident light for each unit pixel, each of said unit pixels comprising:
a solution which includes charged particles which are moved by electrophoresis and intercept a progress of the incident light; and
electrodes for applying an voltage to the particles in order to adjust the transmissivity of the incident light by changing the arrangement of the particles.

2. The mask for forming biomolecules or polymer array as claimed in claim 1,
wherein said solution is a suspending fluid consisted of a fluid and a plurality of charged particles.

3. The mask for forming biomolecules or polymer array as claimed in claim 2,
wherein said fluid comprises any material selected from the group consisting of a fluorocarbon, chlorocarbon, and fluorochlorocarbon.

4. The mask for forming biomolecules or polymer array as claimed in claim 2,
wherein said particles comprise any material selected from the group consisting of barium sulfates, kaolins, zink oxides and TiO₂ and said particle size is 500 to 3000 Å.

5. The mask for forming biomolecules or polymer array as claimed in claim 1,
wherein said solution further comprises dispersing agents and said dispersing agents is selected from the group consisting of ethylene glycols, a polyhydroxy compound capable of containing polypylene glycol, 3-amino-i propanol, an amino alcohol compound capable of containing triethanolamine, surface modifiers and charge control agents.

6. The mask for forming biomolecules or polymer array as claimed in claim 1,
wherein said charged particles intercept a progress of an UV light by absorbing or reflecting the UV light.

7. The mask for forming biomolecules or polymer array as claimed in claim 1,
wherein said charged particles are collected on selected electrodes by the voltage difference of said electrodes, and
the incident light is intercepted when the charged particles are collected on the electrodes vertical to the incident direction of said incident light, and
the incident light is transmitted when the charged particles are collected on the electrodes parallel to the transmissive direction of said incident light.

8. The mask for forming biomolecules or polymer array as claimed in claim 1,
wherein said charged particles are collected on selected electrodes by the voltage difference of said electrodes, and
the incident light is transmitted when the charged particles are collected on the electrodes parallel to the transmissive direction of said incident light, and
when the difference voltage of said electrodes does not exists, the charged particles are randomly distributed to intercept the incident light.

9. A method for forming biomolecules or polymer array using the programmable mask as claimed in claim 1, said method comprising steps of:
(a) irradiating a selected area of the molecules which are fixed on the surface of the mask and have a protective group with UV lights using said programmable mask; and
(b) flowing a solution containing the biomolecules or polymer monomer which needs to be fixed into said selected area of the molecules.

10. A method for forming biomolecules or polymer array using the programmable mask as claimed in claim 2, said method comprising steps of:
(a) irradiating a selected area of the molecules which are fixed on the surface of the mask and have a protective group with UV lights using said programmable mask; and
(b) flowing a solution containing the biomolecules or polymer monomer which needs to be fixed into said selected area of the molecules.

11. A method for forming biomolecules or polymer array using the programmable mask as claimed in claim 3, said method comprising steps of:
(a) irradiating a selected area of the molecules which are fixed on the surface of the mask and have a protective group with UV lights using said programmable mask; and
(b) flowing a solution containing the biomolecules or polymer monomer which needs to be fixed into said selected area of the molecules.

12. A method for forming biomolecules or polymer array using the programmable mask as claimed in claim 4, said method comprising steps of:
(a) irradiating a selected area of the molecules which are fixed on the surface of the mask and have a protective group with UV lights using said programmable mask; and
(b) flowing a solution containing the biomolecules or polymer monomer which needs to be fixed into said selected area of the molecules.

13. A method for forming biomolecules or polymer array using the programmable mask as claimed in claim 5, said method comprising steps of:
(a) irradiating a selected area of the molecules which are fixed on the surface of the mask and have a protective group with UV lights using said programmable mask; and
(b) flowing a solution containing the biomolecules or polymer monomer which needs to be fixed into said selected area of the molecules.

14. A method for forming biomolecules or polymer array using the programmable mask as claimed in claim 6, said method comprising steps of:
(a) irradiating a selected area of the molecules which are fixed on the surface of the mask and have a protective group with UV lights using said programmable mask; and
(b) flowing a solution containing the biomolecules or polymer monomer which needs to be fixed into said selected area of the molecules.

15. A method for forming biomolecules or polymer array using the programmable mask as claimed in claim 7, said method comprising steps of:
(a) irradiating a selected area of the molecules which are fixed on the surface of the mask and have a protective group with UV lights using said programmable mask; and
(b) flowing a solution containing the biomolecules or polymer monomer which needs to be fixed into said selected area of the molecules.

16. A method for forming biomolecules or polymer array using the programmable mask as claimed in claim 8, said method comprising steps of:
(a) irradiating a selected area of the molecules which are fixed on the surface of the mask and have a protective group with UV lights using said programmable mask; and
(b) flowing a solution containing the biomolecules or polymer monomer which needs to be fixed into said selected area of the molecules.
